(19) United States
(12) Patent Application Publication
(10) Pub. No.: US 2013/0237805 A1
(43) Pub. Date: Sep. 12, 2013

(54) MAGNETIC RESONANCE APPARATUS

(71) Applicants: PETER DIETZ, Furth (DE); Annette Stein, Erlangen (DE)

(72) Inventors: PETER DIETZ, Furth (DE); Annette Stein, Erlangen (DE)

(21) Appl. No.: 13/603,504

(22) Filed: Sep. 5, 2012

(30) Foreign Application Priority Data
Sep. 9, 2011 (DE) 102011082407.3

(51) Int. Cl. A61B 5/055

(52) U.S. Cl. CPC A61B 5/055
USPC 600/410

(57) ABSTRACT

A magnetic resonance apparatus having a magnet unit is provided. The magnet unit includes a main magnet, a gradient coil and a high frequency coil unit, a cylindrical imaging region for imaging a patient. The magnet unit cylindrically surrounds the imaging region, and a housing unit surrounding the magnet unit. The housing unit has a rigid, fixedly installed housing shell unit arranged between the magnet unit and the imaging region. The housing unit has at least one noise protection unit arranged to surround the magnet unit at least partially and to be removable within the imaging region.

### CROSS REFERENCE TO RELATED APPLICATIONS

This application claims priority of German application No. 10 2011 082 407.3 filed Sep. 9, 2011, which is incorporated by reference herein in its entirety.

### FIELD OF INVENTION

The present application relates to a magnetic resonance apparatus having a magnet unit, which includes a main magnet, a gradient coil and a high frequency coil unit, a cylindrical imaging region for imaging a patient, wherein the magnet unit cylindrically surrounds the imaging region, and a housing unit surrounding the magnet unit having a rigid, fixedly installed housing shell unit, which is arranged between the imaging region and the magnet unit.

### BACKGROUND OF INVENTION

During operation, magnetic resonance apparatuses have high noise levels which can cause discomfort for a patient located in an imaging region of the magnetic resonance apparatus for the purposes of an examination. Large vibration amplitudes develop on a surface of a high frequency coil unit, which surrounds the imaging region for the patient, said vibration amplitudes resulting in significant noise radiation for the patient. Acoustic resonances can also develop within the imaging region, said acoustic resonances possibly representing an additional noise load. Conventional magnetic resonance apparatuses comprise a rigid, fixedly installed housing shell unit with a high noise radiation.

### SUMMARY OF INVENTION

The object underlying the present application is to provide a magnetic resonance apparatus, with which an effective noise protection is achieved for a patient in an imaging region. The object is achieved by the features of the independent claims. Embodiments are described in the dependent claims.

The application is based on a magnetic resonance apparatus having a magnet unit, which includes a main magnet, a gradient coil and a high frequency coil unit, a cylindrical imaging region for imaging a patient, wherein the magnet unit cylindrically surrounds the imaging region, and a housing unit surrounding the magnet unit with a rigid, fixedly installed housing shell unit which is arranged between the magnet unit and the imaging region.

It is proposed that the housing unit comprises at least one noise protection unit, which is arranged at least partially surrounding the magnet unit so as to be removable within the imaging region. A shielding of the high frequency coil unit and of the gradient coil in respect of a noise radiation can be achieved. Furthermore, a noise reduction within the imaging region can be achieved for the patient during a magnetic resonance examination, wherein the noise reduction up to 10 dB can be achieved. The noise protection unit comprises a dampening and/or decoupling of sound waves in a frequency range, in which a noise radiation of the magnetic resonance apparatus takes place. In addition, depending on the requirements for a magnetic resonance examination the noise protection unit can also be arranged on the magnetic resonance apparatus within the imaging region, and/or fastened thereto, and the use of the noise protection unit is thus dispensed with for instance for a magnetic resonance examination in the case of a claustrophobic patient. In this context, a noise protection unit which is arranged so as to be removable is understood to mean a noise protection unit, which, depending on the requirements for a magnetic resonance examination, can be arranged so as to protect the patient from a large noise load within the imaging region on the magnetic resonance apparatus, such as on the rigid, fixedly installed housing shell unit and/or on a patient couch of the magnetic resonance apparatus, by an operator, such as for instance clinical staff attending to the magnetic resonance apparatus, wherein a mode of operation of the noise protection unit and/or the magnetic resonance apparatus is retained during the removal or disassembly of the noise protection unit. The rigid, fixedly installed housing shell unit may be formed for instance by a supporting construction of the high frequency coil unit, such as for instance made of a fiber-reinforced plastic. Alternatively, the rigid, fixedly installed housing shell unit may also be formed by a housing shell unit embodied separately from the high frequency coil unit.

Furthermore, it is proposed that the noise protection unit comprises at least one absorption unit, as a result of which an absorption of air-borne sound waves can be achieved. A dampening of air-borne sound waves, such as in a frequency range of up to 1500 Hz, such as up to 1000 Hz, takes place by the absorption unit, wherein the air-borne sound waves propagate along a surface of the rigid, fixedly installed housing shell unit. In this context, an absorption unit is understood to mean a sound-absorbing absorption unit, which is configured to convert sound energy from sound waves into an oscillation energy of inaudible oscillation waves, and accordingly to reduce or prevent a reflection of audible sound waves onto a boundary surface. The sound waves, such as the air-borne sound waves, herewith excite individual particles, such as for instance foam particles, of the absorption unit to oscillate, wherein a generated oscillation energy is converted into thermal energy within the absorption unit. Oscillation energy is taken from the sound waves, such as the air-borne sound waves and the air-borne sound waves are dampened. A layer thickness of the absorption unit may amount here from approximately 5 mm to a few cm. The absorption unit may be formed from all materials appearing meaningful to the person skilled in the art, such as for instance a melamine material and/or a porous material with open pores.

The noise protection unit comprises at least one spring-mass unit, as a result of which a radiation of sound waves from the rigid, fixedly installed housing shell unit into the imaging region can be reduced. A reduction in the noise radiation on account of the spring mass unit takes place here with an increased frequency of the sound waves. In this context, a spring-mass unit is understood to mean a unit of the noise protection unit, which has at least one elastic spring element and at least one heavy mass element, so that on account of a high mass inertia and an absorption of oscillation energy from sound waves, a dampening and/or decoupling of sound waves is achieved. The heavy mass element comprises a surface density of at least 3 kg/m² or at least of 5 kg/m² auf. The at least one elastic spring element and the at least one heavy mass element may herewith be embodied in one piece.

In a development of the application, it is proposed that the noise protection unit has at least one facing shell unit. A radiation of sound waves into the imaging region can be reduced by a reflection of sound waves taking place at least partially on the facing shell unit. The facing shell may simultaneously be the unit of the housing unit which is visible to the patient and fulfill such sterility requirements which are needed for a clinical measuring operation. The facing unit may also be integrated within the spring mass unit as a heavy mass element.

It is further proposed that the noise protection unit is arranged at least partially at a distance from the rigid, fixedly installed housing shell unit, as a result of which an effective absorption of air-borne sound can be achieved on account of reducing sound pressure. In addition, the distance between the noise protection unit and the rigid, fixedly installed housing shell unit achieves a softness of an acoustic spring in the spring mass unit and in such a way a dampening property is achieved in respect of a radiation of sound waves into the imaging region.

A compact housing unit can be achieved if the noise protection unit is arranged in an arched manner on the rigid, fixedly installed housing shell unit. The noise protection unit which can additionally be introduced into the imaging region can be minimized in a spatial context so that a large region is available to the patient within the imaging region. The arched noise protection unit may herewith cover a region in respect of a noise radiation of the rigid, fixedly installed housing shell unit, which is arranged within a region which is enclosed by a couch surface of the patient couch and the rigid housing shell unit.

In an alternative embodiment of the application, the noise protection unit may also comprise a cylindrical shape, as a result of which a region of the imaging region, which is not occupied by the patient, can be filled by the noise protection unit. A cross-section of the imaging region can herewith be filled by the noise protection unit without in the process negatively affecting the patient in a spatial context and the patient thus being protected from a high noise load.

Furthermore, it is proposed that the housing unit comprises at least one fastening unit for fastening the noise protection unit to the rigid, fixedly installed housing shell unit, as a result of which a direct fastening can be achieved within the imaging region on the housing shell unit. The fastening unit is provided for a detachable fastening of the noise protection unit, so that the noise protection unit can be disassembled from the housing shell unit in a structurally simple manner at any time without negatively affecting its mode of operation. The fastening unit may for instance include clamping elements, such as flexible clamping elements, which provide for a secure hold of the noise protection unit on the housing shell unit.

It is also proposed that the housing unit comprises at least one fastening unit for fastening the noise protection unit on a patient couch of the magnetic resonance apparatus, as a result of which an unused sub region of the imaging region can be occupied with the noise protection unit when positioning the patient within the imaging region. The fastening unit may be arranged here for instance on a front side, which is firstly inserted into the imaging region, of the patient couch. Alternatively, the noise protection unit can also be positioned manually by an operator directly within the imaging region.

The noise protection unit is embodied to be compatible in terms of magnetic resonance. An embodiment which is compatible in terms of magnetic resonance is understood here to mean that the noise protection unit is formed from a non-magnetizable material.

### BRIEF DESCRIPTION OF THE DRAWINGS

Features and details of the application result from the embodiments described below as well as with the aid of the drawings, in which:

FIG. 1 shows a disclosed magnetic resonance apparatus in a schematic representation,

FIG. 2 shows a sub region of the magnetic resonance apparatus having a first embodiment of a noise protection unit and

FIG. 3 shows a sub region of the magnetic resonance apparatus having a second embodiment of a noise protection unit.

### DETAILED DESCRIPTION OF INVENTION

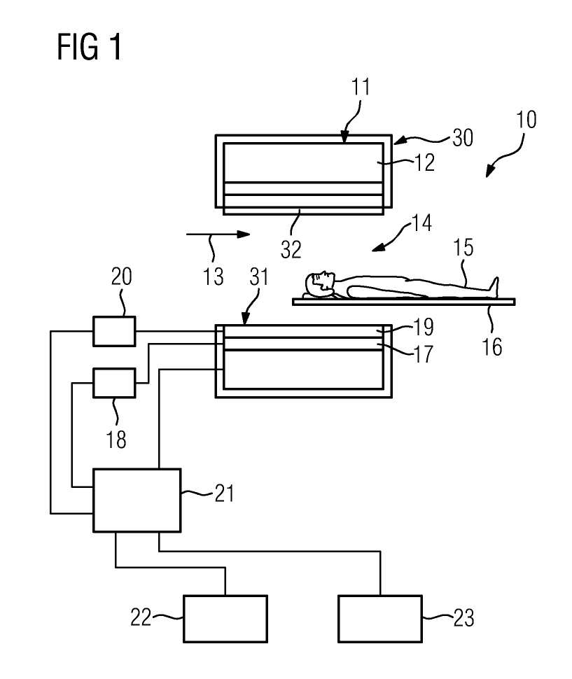

FIG. 1 shows a schematic representation of a disclosed magnetic resonance apparatus 10. The magnetic resonance apparatus 10 includes a magnet unit 11 with a main magnet 12 to generate a strong and constant main magnetic field 13. In addition, the magnetic resonance apparatus 10 comprises a cylindrical imaging region 14 for imaging a patient 15, wherein the imaging region 14 is surrounded by the magnet unit 11 in a circumferential direction. The patient 15 can be introduced into the imaging region 14 by a patient couch 16 of the magnetic resonance apparatus 10. The patient couch 16 is to this end arranged so as to be moveable within the magnetic resonance apparatus 10. Furthermore, the magnetic resonance apparatus 10 comprises a housing unit 30 surrounding the magnet unit 11.

The magnet unit 11 further comprises a gradient coil 17 for generating magnetic field gradients, which is used for spatial encoding during an imaging process. The gradient coil 17 is controlled by a gradient control unit 18. Furthermore, the magnet unit 11 comprises a cylindrical high frequency coil unit 19 and a high frequency control unit 20 for exciting a polarization, which develops in the main magnetic field 13 generated by the main magnet 12. The high frequency coil unit 19 is controlled by the high frequency control unit 20 and radiates high frequency magnetic resonance sequences into an examination room, which is formed by the imaging region 14. The magnetization is herewith deflected from its position of equilibrium. Furthermore, magnetic resonance signals are received by the high frequency coil unit 19.

The magnetic resonance apparatus 10 comprises a control unit 21 formed from a computing unit in order to control the main magnet 12, the gradient control unit 18 and to control the high frequency control unit 20. The computing unit controls the magnetic resonance apparatus 10 centrally, such as for instance implementing a predetermined imaging gradient echo sequence. Control information such as for instance imaging parameters, and reconstructed magnetic resonance images can be displayed on a display unit 22, for instance on at least one monitor, of the magnetic resonance apparatus 10 for an operator. The magnetic resonance apparatus 10 also comprises an input unit 23, by which information and/or parameters during a measuring process can be input by an operator.

The magnetic resonance apparatus 10 shown can naturally include further components, which magnetic resonance apparatuses 10 usually comprise. A general functionality of a magnetic resonance apparatus 10 is also known to the person skilled in the art, so that a detailed description of the general components is omitted.

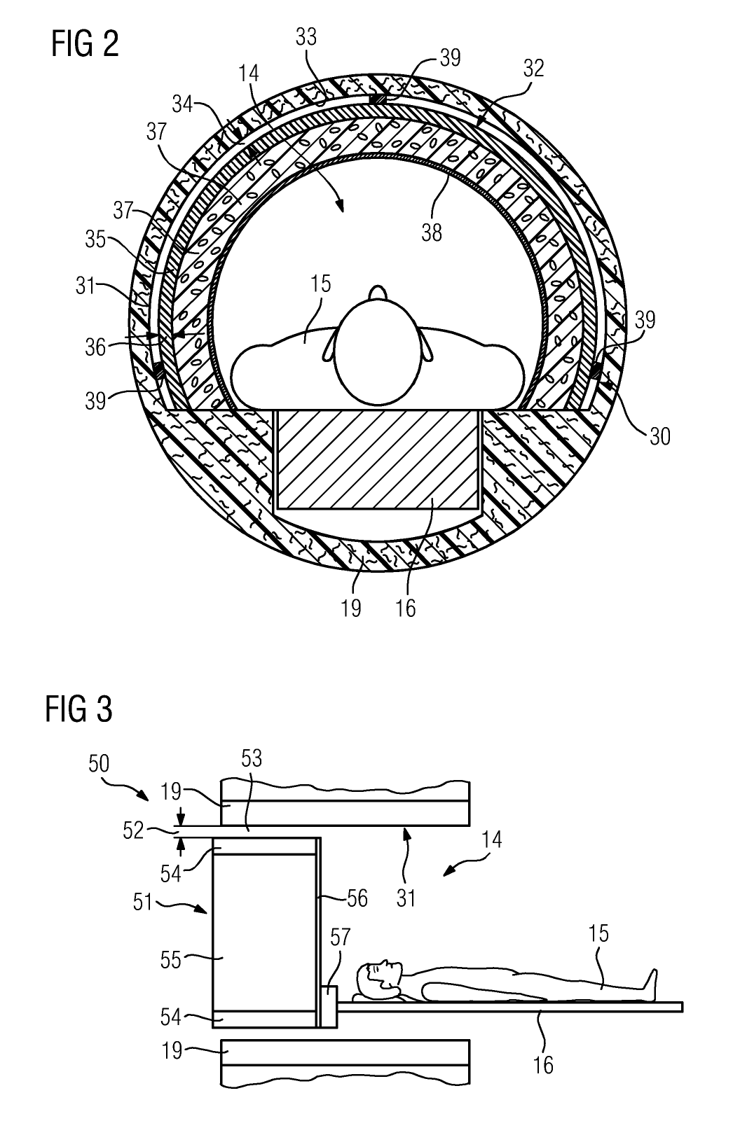

FIG. 2 shows the housing unit 30 in closer detail. The housing unit 30 comprises a rigid, fixedly installed housing shell unit 31, which, in the present embodiment is formed of a support unit, which is formed for instance of a glass fiber-reinforced plastic, of the high frequency coil unit 19. The rigid, fixedly-installed housing shell unit 31 is in this way formed of a side of the support unit facing the imaging region 14 and is provided for instance with a lacquer.

Moreover, the housing unit 30 comprises a noise protection unit 32, which, in the present embodiment, comprises an arched cross-sectional surface and is arranged on a side 33 of the housing shell unit 31 facing the imaging region 14. The noise protection unit 32 is herewith arranged hereupon at a distance 34 from the housing shell unit 31. A maximum distance 34 of the noise protection unit 32 from the housing shell unit 31 may amount here to 5 cm, however, the maximum distance 34 is arranged between 2 cm and 3 cm. To this end, the housing unit 30 comprises spacing elements (not shown), which are arranged between the housing shell unit 31 and the noise protection unit 32. Alternatively, it is also possible for the noise protection unit 32 to be arranged directly, such as without a spacing, on the side 33 of the housing shell unit 31 facing the imaging region 14.

In the present embodiment, the noise protection unit 32 includes three different noise reduction units. A first noise reduction unit is formed by an absorption unit 35, which is formed from a sound-absorbing material, such as for instance a melamine material and/or a porous material. A layer thickness 36 of the absorption unit 35 can amount here to be between 5 mm and a maximum of 5 cm, however a maximum of 3 cm. The absorption unit 35 is arranged on a side of the noise protection unit 32 facing the housing shell unit 31, so that a dampening of air-borne sound waves, which, during operation of the magnetic resonance apparatus 10, propagate along the housing shell unit 31, takes place.

A second noise reduction unit of the noise protection unit 32 is formed by a spring-mass unit 37, which includes an elastic spring element as an acoustic spring and a heavy mass element as an acoustic mass. The spring-mass unit 37 is arranged on a side of the absorption unit 35 facing the imaging region 14. During operation of the magnetic resonance apparatus 10, sound waves radiated by the housing shell unit 31 are dampened by the spring-mass unit 37.

A third noise reduction unit of the noise protection unit 32 is formed by a facing shell unit 38, which is arranged on a side of the spring-mass unit 37 facing the imaging region 14. The facing shell unit 38 can be formed rigidly in this way with a high mass and/or high density, so that a reflection of sound waves, which are radiated from the housing shell unit 31 in the direction of the imaging region 14, is achieved. Moreover, the facing shell unit 38 can also be formed by a flexible layer, such as for instance a soft mass layer, such as an artificial leather layer and/or a washable material layer, and be included in the spring-mass unit 37. A surface density of the facing shell unit 38 may amount to at least 3 kg/m² or to at least 5 kg/m².

In an alternative embodiment of the noise protection unit 32, the absorption unit 35 can be embodied as an acoustic spring of the spring-mass unit 37. Moreover, it is also conceivable that the noise protection unit 32 comprises only one or two of the three noise reduction units for an effective noise reduction and at least one of the noise reduction units is dispensed with in an embodiment of the noise protection unit 32.

The noise protection unit 32 is also embodied to be compatible in terms of magnetic resonance.

The housing unit 30 comprises a fastening unit 39 for arranging the noise protection unit 32 within the imaging region 14. The fastening unit 39 is herewith provided for a detachable fastening of the noise protection unit 32, so that the noise protection unit 32 can be arranged and/or fastened by an operator at any time within the imaging region 14 on the housing shell unit 31 or can be removed from the housing shell unit 31. The housing shell unit 31 can be fastened here without the need for tools.

For fastening purposes, the fastening unit 39 in the present embodiment comprises clamping elements (not shown in more detail) which are arranged on the noise reduction unit 32 and/or on the housing shell unit 31. The noise reduction unit 32 can be arranged and/or fastened to the housing shell unit 31 by the clamping elements in a rapid fashion and if necessary, such as with a claustrophic patient for instance, can be disassembled from the housing shell unit 31 and removed from the imaging region 14 quickly, without herewith negatively affecting the mode of operation of the noise reduction unit 32.

Alternatively, further fastening units which appear meaningful to the person skilled in the art, which enable a detachable fastening of the noise protection unit 32 to the housing shell unit 31, are conceivable at any time, such as for instance a pulling tool with a cable and/or clamping of the noise reduction unit 32 with the housing shell unit 31, wherein a clamping force between the noise reduction unit 32 and the housing shell unit 31 can be produced on account of a clamped form of the noise reduction unit 32.

A cladding of the housing shell unit 31 with the noise protection unit 32 brings about a shielding of the high frequency coil unit 19 and of the gradient coil 17 in respect of noise radiation in the direction of the imaging region 14 during operation of the magnetic resonance apparatus. In this way a noise reduction of up to 10 dB for the patient is achieved within the imaging region 14.

FIG. 3 shows an alternative embodiment of the housing unit 50. Components, features and functions which remain the same are basically numbered with the same reference characters. The description below is restricted to the differences from the embodiment in FIGS. 1 and 2, wherein with respect to the components, features and functions which remain the same, reference is made to the description of the embodiment in FIGS. 1 and 2.

FIG. 3 shows an alternative embodiment of the housing unit 50. The housing unit 50 comprises a housing shell unit 31, which is embodied in a similar manner to the embodiment in FIG. 2. Moreover, the housing unit 50 comprises a noise protection unit 51, which is embodied cylindrically in the present embodiment, so that a cross-section of the imaging region 14 of the magnetic resonance apparatus 10 is filled by the noise protection unit 14 within the sub region of the imaging region 14 which is not required by the patient 15. The noise protection unit 51 is however arranged at a distance 52 of approx. 2 cm to 3 cm from the housing shell unit 31 so that a free air flow channel 53 is provided. Moreover, the noise protection unit 51 for a patient monitoring can be formed at least partially from optically transparent materials.

The noise protection unit 51 herewith likewise includes an absorption unit 54, a spring-mass unit 55 and a facing shell unit 56. The facing shell unit 56 is herewith arranged on a side of the noise protection unit 51 facing the patient 15.

To fasten the noise protection unit 51, the housing unit 50 comprises a fastening unit 57, which enables the noise protection unit 51 to be fastened on a patient couch 16 of the magnetic resonance apparatus 10. The noise protection unit 51 is herewith fastened on an end region of the patient couch 16 in a removable manner, wherein the end region is firstly inserted into the imaging region 14 upon insertion of the patient couch 16 into the imaging region 14. The fastening unit 57 to this end comprises fastening elements (not shown), which may include conventional, detachable fastening elements, such as for instance latching elements and/or clamping elements. It is also conceivable for the noise protection unit 51 to be positioned and arranged within the imaging region 14 by a transport unit in order to transport the patient couch 16.

Alternatively, it is to this end also conceivable for the noise protection unit 51 to be fastened on the housing shell unit 31 within the imaging region 14, such as for instance by a clamping unit. It is moreover also conceivable for the same to be easily inserted into the imaging region 14 by an operator of the magnetic resonance apparatus 10 for an arrangement of the noise protection unit 51 within the imaging region 14.

1. A magnetic resonance apparatus, comprising:
an imaging region for imaging a patient;
a magnet unit cylindrically surrounding the imaging region; and
a housing unit surrounding the magnet unit,
wherein the magnet unit comprise a main magnet, a gradient coil, and a high frequency coil unit,
wherein the housing unit comprises a rigid, fixedly installed housing shell unit that is arranged between the magnet unit and the imaging region, and
wherein the housing unit comprises a noise protection unit that is arranged to surround the magnet unit at least partially and to be removable within the imaging region.

2. The magnetic resonance apparatus as claimed in claim 1, wherein the noise protection unit comprises at least one absorption unit.

3. The magnetic resonance apparatus as claimed in claim 1, wherein the noise protection unit comprises at least one spring-mass unit.

4. The magnetic resonance apparatus as claimed in claim 1, wherein the noise protection unit comprises at least one facing shell unit.

5. The magnetic resonance apparatus as claimed in claim 1, wherein the noise protection unit is arranged at least partially at a distance from the rigid, fixedly installed housing shell unit.

6. The magnetic resonance apparatus as claimed in claim 1, wherein the noise protection unit is arranged arched on the rigid, fixedly installed housing shell unit.

7. The magnetic resonance apparatus as claimed in claim 1, wherein the noise protection unit comprises a cylindrical shape.

8. The magnetic resonance apparatus as claimed in claim 1, wherein the housing unit comprises at least one fastening unit for fastening the noise protection unit to the rigid, fixedly installed housing shell unit.

9. The magnetic resonance apparatus as claimed in claim 1, wherein the housing unit comprises at least one fastening unit for fastening the noise protection unit on a patient couch of the magnetic resonance apparatus.

10. The magnetic resonance apparatus as claimed in claim 1, wherein the noise protection unit is compatible of magnetic resonance.
